(19) United States
(12) Patent Application Publication
(10) Pub. No.: US 2006/0222600 A1
(43) Pub. Date: Oct. 5, 2006

(54) Oral care regimens and devices

(75) Inventor: Aleksey Mikhailovich Pinyayev, West Chester, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/378,496

(22) Filed: Mar. 17, 2006

Related U.S. Application Data
(60) Provisional application No. 60/667,499, filed on Apr. 1, 2005.

(51) Int. Cl. A61K 8/00

(52) U.S. Cl. 424/49

(57) ABSTRACT

Disclosed are various oral care devices and methods. One method includes introducing a composition into the oral cavity, wherein the composition includes a reactive species generating agent. Output from a light emitting element is directed at a portion of the soft tissue of the oral cavity for a period of time sufficient to generate one or more reactive species from the reactive species generating agent.

### CROSS REFERENCE TO RELATED APPLICATION

This application claims the benefit of U.S. Provisional Application No. 60/667,499 filed on Apr. 1, 2005, the substance of which is incorporated herein by reference.

### FIELD OF THE INVENTION

The present invention relates to oral care regimens and devices which can be used to affect microorganisms in an oral cavity.

### BACKGROUND

Efforts have been made in the oral health care field to provide therapeutic methods beyond conventional brushing with a manual toothbrush and a dentifrice. For example, a wide array of electrically powered toothbrushes are commercially available which can provide superior benefits over manual brushes. In addition, toothbrushes are available that feature bristles or brushing elements adapted for specific types of consumers, such as children.

Furthermore, a variety of dentifrice products are available, many of which are designed for specific functions such as whitening of dental surfaces. Moreover, numerous other oral care products are available for various conditions or treatments such as mouthwashes, bleaching strips, and flosses.

Although satisfactory in many respects, a need remains for further advances and improvements in oral health care, and specifically, in therapeutic practices performed by consumers of oral health care products.

### SUMMARY OF THE INVENTION

In one aspect, the present invention provides a method of affecting one more types of microorganisms in an oral cavity. The method comprises introducing a composition into the oral cavity and exposing the oral cavity to energy which activates the composition to produce one or more reactive species. The method may also comprise brushing the teeth with a dentifrice and a toothbrush comprising a light-emitting element.

### BRIEF DESCRIPTION OF THE DRAWINGS

The invention may take physical form in certain parts and arrangements of parts, embodiments of which will be described in detail in this specification and illustrated in the accompanying drawings which form a part hereof, and wherein:

FIG. 1 is a perspective view of an electric toothbrush in accordance with the present invention;

FIG. 2 is a top plan view of the electric toothbrush of FIG. 1;

FIG. 3 is a cross-sectional side view of the toothbrush of FIG. 2, taken along line 3-3 thereof;

FIG. 4A is an end view of a toothbrush head of the present invention during use;

FIG. 4B is an end view of a toothbrush head of the present invention during use;

FIGS. 5A to 5C are partial bottom plan views of a toothbrush head of the present of invention;

FIG. 6 is a perspective view of a tongue device of the present invention; and

FIG. 7 is a bottom plan view of the tongue device of FIG. 6.

### DETAILED DESCRIPTION OF THE EMBODIMENTS

Generally, the present invention relates to oral care regimens utilizing one or more oral care devices or appliance(s) and one or more oral care composition(s). The oral care appliance(s) used in the regimens described herein may comprise a light-emitting element or other energy emitting element (e.g., a heat emitting element) that may activate an oral care composition. The oral care appliances may be provided in the form of an electric toothbrush and may also, or instead, include a tongue device or structure. As used herein, the phrase “tongue device or structure” is intended to refer to devices or structures that are adapted for use on or with the tongue. The oral care compositions can include one or more reactive species generating (RSG) agents that, upon exposure to an energy input such as light, generate one or more reactive species. Reactive species refer to unstable atoms or molecules which may easily react with other atoms or molecules to from new species. Reactive species may include radicals and excited atoms, such as singlet oxygen. Examples include hydroxyl radicals, hydroperoxy radicals, alkyl radicals, and alkoxy radicals, and superoxide. Species formed by autoxidation of reactive species are also included. The reactive species can adversely affect one or more microorganisms in the oral cavity. In one embodiment, the regimens are directed to killing, treating, reducing, or adversely affecting bacteria in the oral cavity and, in another embodiment, treating, reducing, or adversely affecting bacteria associated with the soft and/or hard tissue of the oral cavity. As used herein, the phrase “soft tissue” is intended to encompass one or more of the tongue, papilla, and gingivia, which may include the marginal gingival, gingival suculus, inter dental gingival, gingival gum structure on lingual and buccal surfaces up to and including muco-gingival junction and pallet. Other soft tissue within the oral cavity is also within the scope of the present invention, including, for example, oral tissue of the cheek.

The term “oral care appliances” or “oral care devices” may include any appliance or device adapted for use in or having an effect in an oral cavity, including but not limited to toothbrushes, tongue devices, polishers, gum massagers, flossing instruments, trays, applicators, mouth guards and other hand-held devices. The term “oral care composition” (or substance) as used herein refers to a composition that provides one or more oral care benefits to the user. Non-limiting forms of oral care compositions include dentifrices, gels, rinses, tablets, strips, paint-on formulations, foaming formulations, films, quick dissolving strips or films, and the like. Activation can occur upon exposure of the RSG agent to one or more of light, heat, electrical energy, acoustic energy, vibrational energy, or other energy source. Although the present invention is in no way limited to toothbrushes and tongue devices only but rather may be reapplied to various other oral care devices, various aspects of the present invention will now be described with respect to toothbrushes and tongue devices for ease of illustration.

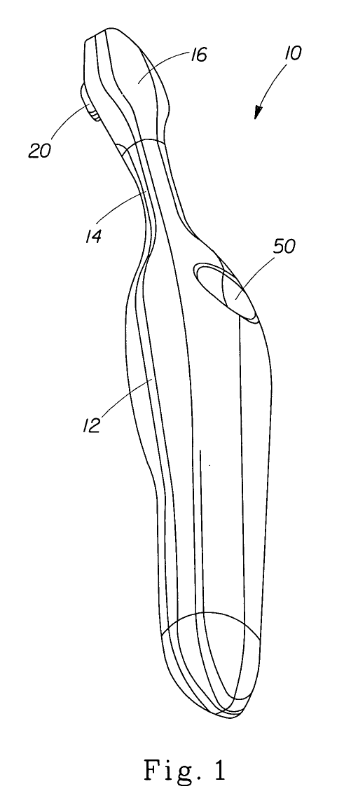

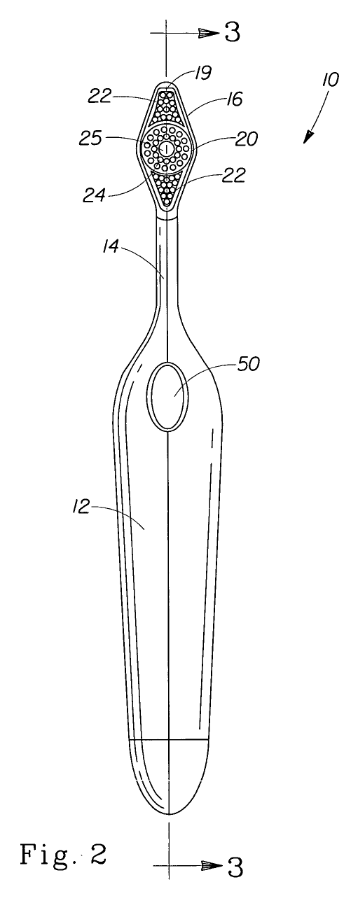

As shown in FIGS. 1 and 2, an electric toothbrush includes a handle 12 and a neck 14 attached to the handle 12. A head 16 is attached to neck 14. Typically, the head is larger than the neck 14, which is also typically smaller than the handle 12. Referring now to FIG. 2, the head 16 further is defined by a longitudinal axis 19, and may comprise one or more moving bristle holders 20 and one or more optional static bristle holders 22. In this embodiment the static bristle holders 22 are located on opposite sides of the moving bristle holder 20. The moving bristle holder 20 in this embodiment is located at the center of the head 16, but it may be located anywhere on the head. The moving bristle holder 20 includes a plurality of bristles 24 supported and retained on the bristle holder 20. The moving bristle holder can oscillate or rotate about an axis of motion approximately normal to the longitudinal axis 19 of the head 16, although other motions may be provided. The static bristle holders and the arrangement of the static bristles disposed thereon can also be widely varied. For example, the static bristles might partially or wholly circumscribe the moving bristle holders or may be disposed in a gap between the moving bristle holders. Examples of some bristle holder motions and bristle arrangements suitable for use with the present invention are described in US 20030126699; US 20030084525; US 20030084524; US 20030084526; and WO 03/063723; and WO 03/063722.

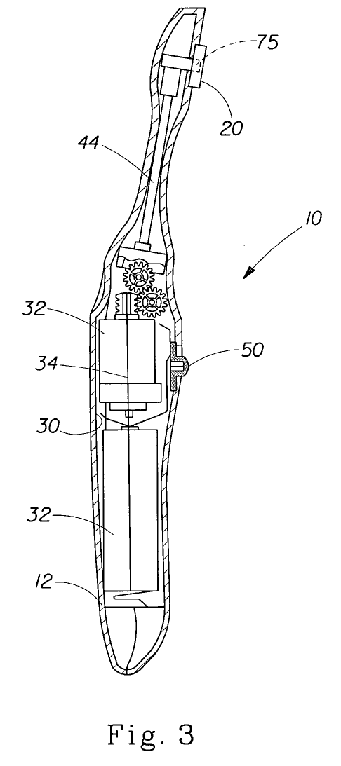

As shown in FIG. 3, the handle 12 further includes a hollow portion 30 which houses a motor 32, and has a longitudinal axis 34. The motor 32 powers the moving bristle holder 20 through a shaft 44. The shaft may rotate, oscillate, linearly reciprocate, gyrate, orbit, or move in a conical fashion when driven by the motor in order to impart one or more motions to the moving bristle holders. A gearing arrangement may operatively interconnect the shaft 44 and the motor 32, although arrangements that do not include gearing arrangements may also be provided. Exemplary shaft and/or gearing arrangements suitable for use with the present invention are shown in U.S. Pat. Nos. 6,360,395 and 5,617,601, and U.S. Patent Application Nos. 2003/0134567 and 2003/0163881 as well as in other patents and patent publications referenced herein. The handle also has a power source, such as one or more batteries, disposed therein for powering the motor and other electrical elements of the toothbrush. Alternatively, the electric toothbrush may be connected to an external power source for powering the motor. One or more switches 50 in electrical communication with the power source may be disposed on the handle for activating the motor and/or the other electrical elements, such as a light emitting element. As used herein, the term “light-emitting” element is intended to refer to an element that converts electrical energy into light, as opposed to an element that merely conducts or transmits light, such as a fiber optic cable or wire (i.e., a light transmitting element). The toothbrush can optionally include a removable head and/or neck that releasably engages the handle. The toothbrushes can also comprise one or more alarms or signaling devices (e.g., a speaker or light source) to indicate for example the beginning, progress, or completion of a particular treatment regimen or process. For instance, an audible signal might indicate the initiation of output from a light emitting element. The alarms or signals can be in the form of auditory, visual, or tactile signals. Tactile signals may include vibration or other motion of certain parts of the toothbrush, for example the handle or the moving bristle holders. Examples of auditory alarms include, but are not limited to one or more beeps, a series of notes, a song of portion thereof, one or more tones, one or more rings, spoken words or phrases, and combinations of these. A timer can also be incorporated into the device so that upon expiration of a set period of time, another or different signal is provided. The timer might be employed to ensure that sufficient exposure to light or one or more oral care compositions has occurred, ensure that exposure times are not exceeded, ensure sufficient time delays between administering of multiple step regimens, indicating to the user to brush or otherwise direct efforts from one region of the oral cavity to another, and combinations of these. For strategies in which it is desired to limit the exposure time for light emission, non-limiting examples of light emission duration include about 1 minute or less or about 30 seconds or less. It is further contemplated that sensors may be incorporated into the toothbrush head that can detect conditions, markers, stimuli, and agents in the oral cavity, such as the presence of bacteria or malodor associated with their presence.

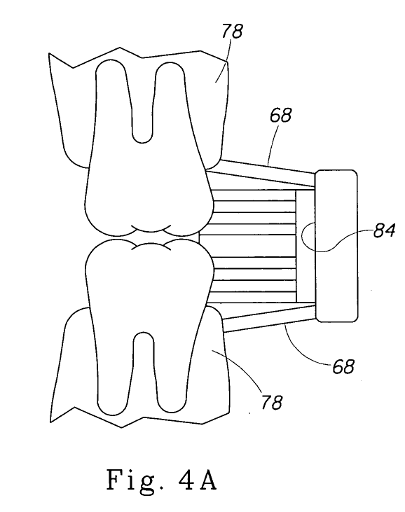

In one embodiment, the toothbrushes of the present invention comprise a light emitting element, and the toothbrush is used in combination with an oral care composition containing an RSG agent that is applied to the soft tissue. The output of the light emitting element is directed to soft tissue of the oral cavity to activate the RSG agent. The output of the light emitting element can be directed at the soft tissue either by the arrangement of the light emitting element on the toothbrush or by the regimen employed by a user. Alternatively, bristles which can optically transmit light may be utilized, particularly where the bristles are arranged to contact the gums or where the bristles are coated so that substantially all of the light is emitted from the bristle tips as opposed to along the bristle length. This can focus the light more effectively upon the interdental spaces between teeth where bacteria may be located. In this arrangement, the circumference of the outer surface of the bristles may be opaque or substantially so while the bristle tips or end portions are transparent or translucent. In one embodiment, at least about 20%, 30%, 40%, 50%, 75%, or 100% of the outer surface is opaque or substantially so. The bristles can be provided with an opaque coating on the outer surface or comprise a transparent or translucent core surrounded by an opaque sheath. The latter bristles can be co-extruded. A co-extrusion process that may be suitable for forming these bristles is described in U.S. Pat. Nos. 6,862,771; 5,313,909; and 5,770,307. In one embodiment, these bristles may be disposed along the sides of a brush head and form an acute angle with the top surface of a brush head so that the bristles can contact the gingiva during use, as shown by way of example in FIG. 4A wherein bristles 68, gingival soft tissue 78 and top surface 84 of a toothbrush head are illustrated.

The oral care composition can be applied to the soft tissue of the oral cavity before, during, or after a traditional tooth brushing regimen. For instance, the oral care composition containing the RSG agent can be applied before or after a step of brushing the teeth with a dentifrice. Alternatively, the RSG agent may be incorporated into the dentifrice and the RSG agent can be activated at the same time as the toothbrushing step occurs.

As used herein, the term “light” is intended to encompass the spectrum of both visible and non-visible (e.g., ultraviolet and infra-red) light. In one embodiment of the toothbrush of the present invention the light emitted from the light-emitting element can be from about 370, 390, 410, 430, 450, 470, 490, 510, 530, 550, 570, 590, 610, 630, 650, 670, 690, 710, 900, 1100 nm and/or less than about 770, 750, 730, 710, 690, 670, 650, 630, 610, 500, 400 nm. In another embodiment the light emitted can have a wavelength of greater than about 420, 430, 440, 450, 460, 470, 480, and/or 490 nm and/or less than about 490, 480, 470, 460, 450, 440, 430 nm. In yet another embodiment the light emitted can have a wave length from about 420, 430, 440, 450, 460, 470 nm and/or less than about 470, 460, 450, 430 nm. It will be appreciated that the particular range of wavelengths selected can depend upon the desired color of the light. The oral care appliance can also emit light of a particular intensity. Intensity can be either luminous intensity measured in candelas (or lumens/steradian), or flux density measured in Watts/meter². In one embodiment the flux density of the inventive illuminated electric toothbrush is from about 20, 30, 35, 40, 45, 50, 55, 60, 70, 100, 200, 250 mW/cm² and/or less than about 300, 250, 200, 150, 100,70, 60, 50, 40, 30 mW/cm² or any combination of these.

Referring to FIG. 4B, a toothbrush 74 is illustrated wherein a light-emitting element is arranged so that it can direct light to soft tissue of the oral cavity during use. In one embodiment, one or more light emitting elements may be provided so that soft tissue, particularly gingival tissue of one or both of the maxillary and mandibular dental arches, may be illuminated by the light emitting elements. In FIG. 4B, two light emitting elements 75 are arranged so that they can illuminate the gingival tissue 78 of both dental arches during the brushing process. Each of the light emitting elements 75 have a principal direction of light emission 80 that forms an acute angle 82 with a top surface 84 of the toothbrush head. In other words, the light-emitting elements 75 can be arranged so that the centerline 80 of the light-emitting 75 element forms an acute angle 82 with the top surface of the head and/or bristle holder. Further, the principle direction of light emission is unobstructed so that the emitted light passes without interference from structures (such as bristles) of the toothbrush head to the soft tissue. The centerline 80 typically passes through the lens or aperture of the light-emitting element. The centerline 80 may form an angle 82 with the top surface of the toothbrush head between about 0, 20, 30, 40, 45, 50, 55 degrees and/or less than about 80, 75, 70, 60, 50, and 40 degrees. When the light-emitting element is disposed within, on, or below a moving and/or static bristle holder, a cylindrical region or volume about the centerline 80 of the light-emitting element can be devoid of bristles so that light is transmitted to the soft tissue without interference from the bristles. It is further contemplated that a brush head or neck of the toothbrush can utilize one or more transparent or translucent panels that allow light emitted from within the brush head or neck to pass therethrough in a manner such that the light is directed at an angle away from the top surface of the toothbrush head. In these embodiments, it is contemplated that the light emitted from the toothbrush head is directed toward the soft tissue to activate an RSG agent while at the same time use of the bristled portion of the toothbrush head provides a more traditional cleaning benefit for the tooth surfaces.

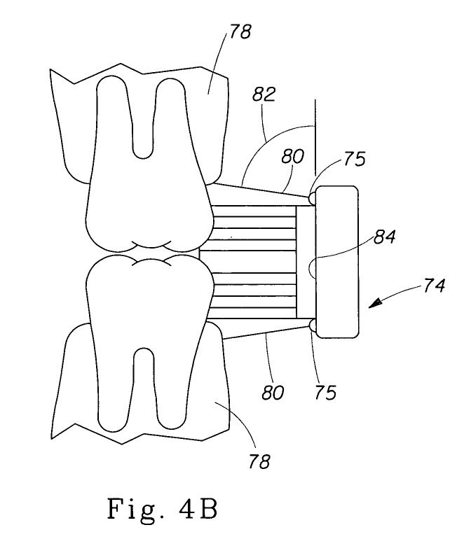

The light-emitting elements can also be arranged so that the principle direction of light emission is generally perpendicular to the top surface of the bristle holders and/or generally parallel to the direction of the bristles of the bristle holder, particularly where it is desirable to provide a tongue device or structure therewith. One example is illustrated in FIGS. 2 and 3, where a light emitting element 75 is disposed in a moving bristle holder 20. The light emitting element 75 can also be arranged at other locations on the head so that the output of the light emitting element can illuminate the tongue when the head is maneuvered into a position where the light is directed onto the tongue. In another embodiment shown in FIGS. 5A to 5C, light may be emitted from light emitting element 75 disposed on a rear surface 86 of the toothbrush head so that it may be directed toward the tongue of a user where bacteria and other microorganisms that can cause malodor may be located. Panels that are selectively painted with light blocking coatings can also be used to tailor the manner in which light is emitted from the device or appliance. FIGS. 5A to 5C illustrate one or more tongue structures which are adapted for contacting the tongue. The tongue structures can provide a massaging, scraping, or cleaning benefit. FIG. 5A illustrates a plurality of protrusions the form of upstanding elastomeric walls 88. The walls can be provided in a variety of shapes and sizes and may partially or wholly encircle the light emitting element. FIG. 5B illustrates a plurality of protrusions in the form of hemispherical bumps 90 which may partially or wholly encircle the light emitting element 75. FIG. 5C illustrates a combination of upstanding walls 88 and bumps 90. As will be appreciated, the protrusions and walls can be provided in a variety of shapes, sizes, and materials. In one embodiment, the protrusions are transparent or translucent. A light emitting element, such as an LED, may be placed behind the protrusions so that the protrusions may transmit light directly to the soft tissue during use.

While the preceding examples illustrate light emitting elements that are located on the head of the toothbrush, it is contemplated that the light emitting elements might be located elsewhere. For example, the light emitting elements might be located on in the neck or handle and light transmitting structures, such as optical fibers, transmit the light to the head for light emission from the head.

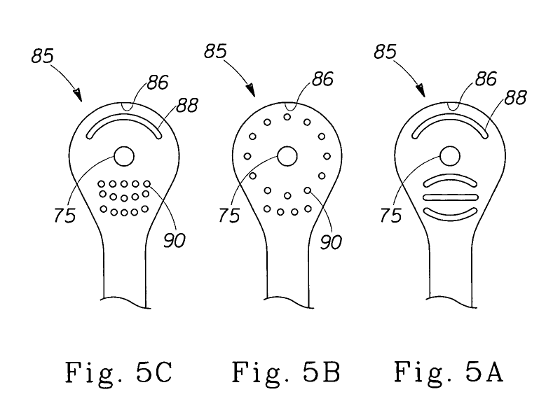

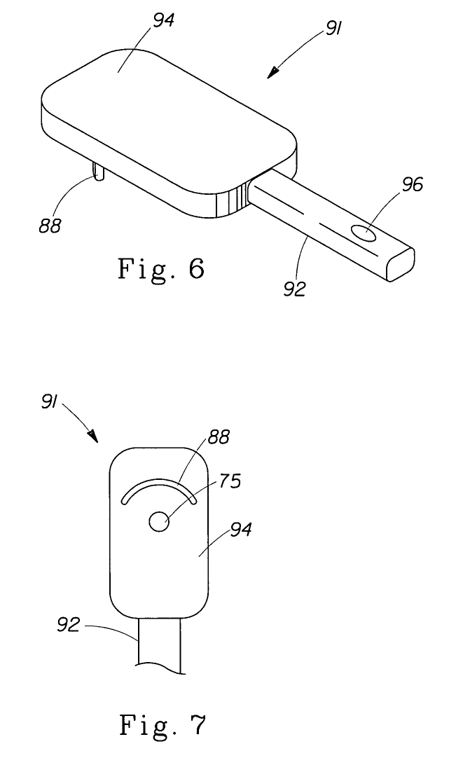

While the tongue structures may be incorporated into a toothbrush head, it is contemplated that the tongue structures may be provided as a separate, stand-alone device, an example of which is shown in FIGS. 6 and 7. The tongue device 91 comprises a handle 92 to which is connected a head 94. The head 94 comprises a light-emitting element 75 and a tongue structure in the form of an upstanding wall 88. The head 94 may be integrally connected to the handle 92 or may be releaseably attached. The handle 92 contains a power source (not shown) for powering the light emitting element 75. A switch 96 may be provided which energizes the light emitting element 75. The tongue device 91 can be manual, or utilize an electrically powered mechanism to move one or more of the protrusions to facilitate scraping or cleaning of the tongue. The tongue devices can utilize interchangeable or walls, protrusions of different sizes, varying quantities of protrusions, etc. Examples of some tongue devices that are suitable for use with the present invention include, but are not limited to, those described in U.S. Pat. Nos. 3,254,356; 2,651,068; 2,405,029; 4,455,704; 4,488,327; 5,217,475; 5,226,197; 5,569,278; 5,735,864; 5,779,475; 5,766,193; 5,893,860; 5,910,151; 5,915,433; 5,916,228; 6,013,089; 5,980,541; 5,984,935; 6,056,763; 6,089,865; 6,099,540; 6,152,939; and 6,440,149. Also of interest is EP 1034721. The tongue structures can be formed from a variety of materials, including elastomeric materials, such as rubbers such as synthetic and natural rubbers. Other materials include polypropylene or polyethylene. The tongue structure can include a flavoring, artificial or natural. The flavoring is preferably included with the plastic prior to fabrication.

Typically, the light-based outputs emit light for a prescribed period of time. For example, light can be used which has a wavelength between about 600 nm and about 900 nm, about 5 to about 10 mW intensity for about 0.5 to about 2 minutes with compositions containing RSG agents. Toluidine blue or methylene blue may be provided in an oral care composition that is applied to the oral cavity for an antibacterial benefit or in a dentifrice used with the toothbrush or dispensed by the toothbrush. Preferably, these agents are used with light having a wavelength of between about 600 nm and about 660 nm. Other RSG agents include vitamins such as riboflavin (vitamin B2) in combination with light having a wavelength between about 410 and about 450, and preferably from about 420 to about 460. Chlorophyll (e.g., chlorophyll a & b, and bacterial chlorophyll) can be used in combination with light having a wavelength between about 400 nm and about 480 nm and specifically at 440 nm, or other radical generating agents such as hydrogen peroxide, urea peroxide, percarbonate and the like at a variety of wavelengths. Metals such as silver, iron, and manganese, while not RSG agents, may be agents that can adversely affect bacteria and other micororganisms if the wavelength of the light results in sufficient heat generation. Light having a wavelength between about 380 nm and about 420 nm may be effective at killing or adversely affecting bacteria and other microorganisms without the use of an RSG agent. Another RSG agent is thioxanthone. This agent can be used in conjunction with light having a wavelength of between about 360 and about 400 nm, and particularly about 380 nm. Furthermore, light in conjunction with one or more vitamins can be utilized in the present invention regimens. Non-limiting examples of vitamins or other corresponding agents include, but are not limited to, riboflavin or vitamin B12. Light having a wavelength of from about 410 to about 450 nm is effective. Riboflavin is a safe agent for oral applications. Dihaematoporphyrin ester and phthalocyamine can also be used. Generally, light having a wavelength between about 600 and about 660 nm, preferably from about 610 to about 650 nm and particularly about 633 nm is effective for these combinations. Additional details are provided in Oral Microbial Immunol., 1993, 8; 182-187. Other RSG agents can include rose bengal; Zn phthalocyanine; porphyrin, in particular hematoporphyrin, uroporphyrin, and tetraphenylporphyrins and their complexes of Zn, Al, Si, Sn, phthalocyanines and their complexes with Zn, Al, Si, Sn and Curcumin.; chlorins, in particular bacterialchlorins; bilirubin; curcumin; EDTA; diethylenetriamine pentacetic acid (DEPTA); NTA; EHDP; ethylenediamine tetra(methylenephosphonic acid); and diethylenetriamine penta(methylenephosphonic acid). RSG agents can be added to the oral care composition in an amount from about 0.1, 0.5, 1, 2, 3, 5, 7, 10 and/or less than about 10, 7, 5, 3, 2, 1, 0.5, 0.1 weight percent, based upon the total amount of the composition. Superoxide may be generated using any of the above sensitizers in combination with an electron donor such as amines and amides—EDTA, DTPA, diethylene triamine pentaphosphonic acid, triethanolamine, triethylamine, tryptophan, tyrosine or acetanilide. In another embodiment nanometer scale zinc diode and titanium dioxide may be used as RSG agents. In some embodiments, multiple RSG agents may be provided in a single oral care composition, wherein each RSG might be activated by a different wavelength of light. Such an arrangement might be useful where each RSG agent is used to target different microorganism species.

In an alternate embodiment, the RSG agent can be produced within the microorganism. Porphyrins, when exposed to light, can produce singlet oxygen and other reactive species. Porphyrins can be naturally formed within the target microorganism or administered separately as an RSG agent. Production of porphyrins within a microorganism can be enhanced by providing porphyrin precursors to the oral cavity. A porphyrin precursor is a compound, molecule, or agent, or combination thereof, which results in the production of porphyrins within a microorganism, such as bacteria, as result of the metabolic activity of the microorganism. The porphyrin precursor is ingested, adsorbed, or absorbed by the microorganism. In one embodiment, the porphyrin precursor is a compound or agent that is used to produce aminolevulinic acid (ALA) and/or porphobilinogen (PBG) within the microorganism. ALA can be later metabolized or transformed into a porphyrin by a process involving one or more cellular enzymes such as ALA synthase, ALA dehydratase, PBG deaminase, uroporphyrinogen III cosynthase, uroporphyrinogen decarboxylase, coproporphyrinogen oxidase, protoporphyrinogen oxidase, and ferrochelatase. An example of a porphyrin precursor is the combination of glycine and succinyl coenzyme A which, when ingested or absorbed by a microorganism, can result in the production of ALA within the target microorganism and eventually a porphyrin. Alternatively, the porphyrin precursor can be ALA, PBG, hydroxymethylbilane (HMB), uroporphyrinogen III, coproporphyrinogen I, or any other agent in the heme biosynthetic pathway for the formation of a porphyrin. These porphyrin precursors can be provided directly to the oral cavity in an oral care composition so that one or both are ingested, absorbed, or adsorbed by the microorganism.

In one embodiment, the porphyrin precursor can be provided in the form of a rinse or dentifrice which is used at least once daily. In another embodiment, the oral care composition containing the porphyrin precursor is administered to the soft and/or hard tissue of the oral cavity between about 1 and about 20 times or between about 5 and about 10 times. The oral care composition containing the porphyrin precursor may be left in the oral cavity at least about 10, 15, 20, 25, 30 or about 60 seconds and/or less than about 5, 4, 3, 2, or 1 minutes. This period may vary depending upon the amount of time for the microorganism to ingest, absorb, or adsorb the porphyrin precursor. Application of light to the oral cavity by an oral care appliance may occur between at least about 10, 15, 20, 25, 30, or about 60 seconds and/or less than about 10, 5, 4, 3, 2, or 1 minutes after the above-described ingestion/absorption/adsorption period. Optionally, an additional step can include removing the oral care composition containing the porphryin precursor after the previously described ingestion/absorption/adsorption time period has lapsed. The oral care composition can be removed by brushing or rinsing with a second oral care composition that does not contain a porphyrin precursor. The oral care composition containing the porphyrin precursor might also be applied to the oral cavity two or more times per day.

Following application of the porphyrin precursor to the oral cavity, light can be applied to the oral cavity to activate the porphyrins. The light can be applied after an incubation period sufficient to result in the production of porphyrins in the microorganism from the porphyrin precursors. The incubation period can be 2, 4, 6, 8, 12, or 24 hours after application of the oral care composition comprising the porphyrin precursor. The incubation period can further extend to 2, 4, 6, 8 12, 14, or more days during which time the oral care composition containing the porphyrin precursor may be applied one or more times per day during this period. Alternatively, the use of an oral care composition comprising a porphyrin precursor followed by application of light with an appropriate wavelength to activate the porphyrins can form part of a daily regimen. The oral care composition can also include a porphyrin precursor as well as other RSG agents.

Porphyrins can absorb light between about 400 nm and about 450 nm, or between about 490 nm and about 550 nm, or between about 580 nm and about 600 nm, and between about 600 nm and about 640 nm, depending upon the porphyrin.

A wide variety of light-emitting elements may be used with the present invention regimens. In one embodiment the lighting-emitting element is a small, low power consumption, light-emitting diode (LED) such as those commercially available under the designation Luxeon™ manufactured by Lumileds Lighting, LLC of San Jose Calif. Other commercially available lighting units include those from American Opto Plus LED Corporation. The LED can operate from a relatively low voltage DC power supply, such as in one embodiment between about 0.5 volt and about 5 volts, an in another embodiment between about 1 volt and 3 volts, and in another embodiment between about 1.6 to about 2.4 volts.

In other embodiments, the light radiation source is solid-state lighting (SSL) including a light-emitting diode (LED) and LED variations, such as, edge emitting LED (EELED), surface emitting LED (SELED) or high brightness LED (HBLED). The LED can be based on different materials such as AlInGaN/AIN (emitting from 285 nm), SiC, AlInGaN, GaAs, AlGaAs, GaN, InGaN, AlGaN, Alln-GaN, BaN, InBaN, AlGalnP (emitting in NIR and IR), etc. LEDs also include organic LEDs which are constructed with a polymer as the active material and which have a broad spectrum of emission. The radiation source can be an LED such as shaping of LED dies, LED with transparent confinement region, photonics crystal structure, or resonant-cavity light-emitting diodes (RCLED).

Other possibilities include a superluminescent diode (SLD) or LED which preferably can provide a broad emission spectrum source. In addition, laser diode (LD), waveguide laser diode (WGLD), and a vertical cavity surface emitting laser (VCSEL) can also be utilized. The same materials used for LED's can be used for diode lasers. Other possibilities include a fiber laser (FL) with laser diode pumping. Fluorescence solid-state light source (FLS) with electro or light pumping from LD, LED or current/voltage sources can also be the radiation source. The FLS can be an organic fiber with electrical pumping.

Lamps such as incandescent lamps, fluorescent lamps, micro halide lamps or other suitable lamps may also be used with the present invention. A lamp can provide the radiation source for white, red, NIR and IR irradiation. For the 5-100 micron range, quantum cascade lasers (QCL) or far infrared emitting diodes can be used. One skilled in the art will appreciate that a variety of radiation sources can provide the necessary optical radiation for the sensor responsive toothbrush depending on size, power requirements, desired treatment regimen, and combinations thereof.

For light-emitting diodes, the dominant or central wavelength can determined by the equations:

$${λ}_{c} = {\int }_{λ min}^{λ max} I ( λ ) \cdot λ \cdot ⅆ l / {\int }_{λ min}^{λ max} I ( λ ) \cdot ⅆ λ$$

For continuous spectrums, and

$${λ}_{c} = {\sum }_{i} {I}_{i} {λ}_{i} / {\sum }_{i} {I}_{i}$$

For discrete spectrums.
Wherein I is illumination intensity and λ is wavelength.

These equations are further described in CIE 127 (1997) entitled “Measurement of LEDs”, which is published by the International Commission of Illumination. These equations and methodology can be also be applied to light-emitting elements other than LEDs, or other methodologies and equations known in the art can be utilized to determine the dominant or central wavelength of a light-emitting element. The spectral (e.g., peak wavelength), photometric (e.g., luminous intensity), radiometric (e.g., radiant intensity), and colormetric (e.g., dominant wavelength) characteristics of the light-emitting elements can be measured using devices known in the art, such as OL 730CV Radiometer/Photometer manufactured by Optronic Laboratories, Inc. of Orlando, Fla. Some light may not have a dominant or central wavelength (e.g., white light).

As previously noted, the term “light” is intended to encompass the spectrum of both visible and non-visible (e.g., ultraviolet and infra-red) light. This spectrum may extend from light having a dominant or centroid wavelength of about 10 nm (far ultraviolet) to light having a centroid wavelength of 106 nm (infrared), or the spectrum may include visible light having a centroid wavelength between about 370 nm and about 770 nm. Further, the spectrum may include visible light having a centroid wavelength between about 370 to about 500. This may be different than the peak wavelength which is the wavelength at which the radiant intensity of the LED is maximum.

The toothbrushes described herein can dispense one or more oral care compositions. For these embodiments, the toothbrushes can utilize a dispensing system that includes one or more cartridges, each containing a particular oral care composition and an RSG agent. Additional details of cartridges, dispensing systems and the like are set forth in U.S. patent application Publication No. 2003/0194678 filed Apr. 25, 2003. Other means for supplying an oral care composition having an RSG agent to the soft tissue include strips, trays, paint on applicators, and the like. Examples of strips which are suitable for use in the inventive method include, but are not limited to, the strips disclosed in U.S. Pat. Nos. 6,096,328, 6,136.297, 6,045,811, 5,989,569, 5,894,017, 5,891,453, 5,879,691, 6,277,458, 6,287,120 and 6,343,932. Examples of trays suitable for use in the inventive method include, but are not limited to, those described in U.S. Pat. Nos. 5,846,058, 5,816,802 and 5,895,218, and other pre-loaded devices such as those described in U.S. Pat. No. 5,310,563. The present invention treatment regimens also include the use of oral care compositions in the form of strips, films, or layers that, when placed within an oral cavity, dissolve. Typically such films are fast dissolving, and dissolve in less than 60 seconds, and often in less than 30 seconds. Additional non-limiting details of such films are provided in U.S. Pat. Nos. 5,948,430 and 6,709,671. The strips, films, or layers can be used by placing the film on the surface of interest, such as for example on a tongue, and then allowing the film to dissolve. Prior to, concurrently, or subsequent to (i) placement of the film on the surface, (ii) dissolving of the film, or (iii) completion of the film dissolving; an oral care appliance can be used as described herein. For instance, a tongue device or toothbrush can be used. Kits can be provided which include one or more of these application means as well as a toothbrush handle or body and a collection of interchangeable head components, each of which can be engaged with the body of the toothbrush.

The oral care compositions herein may also comprise a thickening agent. In one embodiment the thickening agent (or viscosity modifier) can also function to increase retention of the composition on the soft tissue. The viscosity modifier may be present at a level of from about 0.01% to about 20%, in one embodiment from about 0.1% to about 10%, and in another embodiment from about 1% to about 3%, and in yet another embodiment from about 0.4% to about 5%, by weight of the composition. Suitable viscosity modifiers herein include natural and synthetic polymers and gums such as cellulose derivatives (e.g. methylcellulose, carboxymethylcellulose, hydroxyethylcellulose, hydroxypropylcellulose etc), carbomer polymers (e.g. polyacrylic acid copolymer or homopolymer and copolymers of acrylic acid cross linked with a polyalkenyl polyether), karaya gum, guar gum, gelatin, algin, sodium alginate, chitosan, polyethylene oxide, acrylamide polymers, polyvinyl alcohol, polyamines, polyquarternary compounds, ethylene oxide polymers, polyvinylpyrrolidone, cationic polyacrylamide polymers and mixtures thereof. In one embodiment the thickening agent is selected from carbomers, e.g. the class of homopolymers of acrylic acid crosslinked with an alkyl ether of pentaerythritol or an alkyl ether of sucrose. Carbomers are commercially available from B.F. Goodrich as the Carbopol series. In one embodiment the carbopols are Carbopol 934, 940, 941, 956, and mixtures thereof. In another embodiment the viscosity modifier is a hydrophobically modified carbomer. Hydrophobically modified carbomers can increase the retention of compositions herein and/or integral carriers on tooth surfaces and slow the erosion of the compositions once applied on the tooth surfaces. Suitable hydrophobically modified carbomers include acrylate/C10-C30 alkyl acrylate crosspolymer such as Carbopol 1382, Carbopol 1342, Carbopol 1392, and Carbopol ETD 2020, all available from BF Goodrich, and acrylates/C10-C30 alkyl acrylate crosspolymer such as Pemulen TR-1 and Pemulen TR-2 both available from B.F. Goodrich. In one embodiment mixtures of hydrophobically modified carbomers with carbomers can be used. In another embodiment carboxy functional silicones (diacid, monoacid) are used to increase retention of RSG agents on the soft tissue.

Another treatment agent that can be used with the present invention is an optical coupling agent. These compounds provide increased optical access into underlying tissue by reducing the amount of light scattering at the tissue surface. Exemplary optical coupling agents include glycerol; glucose; propylene glycol; polyethylene glycol; polyethylene glycol; x-ray contrasting agents (Trazograph-60, Trazo-graph-76, Verogrann-60, Verografin-76, and Hypaque-60); proteins (hemoglobin, albumin); and combinations thereof. The optical coupling agents can also be used with additives such as ethanol and water (e.g., ethanol, glycerol and water).

In the event light emission and activation or administration of the oral care compositions occur concurrently or substantially so, the present invention includes a strategy in which the dispensed composition passes through a field or beam of emitted light, thereby at least partially activating the composition. As the composition is dispersed within the oral cavity, activation can continue due to exposure to emitted light.

In yet another embodiment, a rinse is used to treat the soft tissue of the oral cavity either prior to and/or after the exposure to the emissions from the electric toothbrush. The rinse comprises an RSG agent and, optionally, a polymer which gives substantivity to the RSG agent, and/or helps adhere to the soft tissue. The teeth are then brushed using the earlier described oral care implement; exposing the soft tissue of the oral cavity to the emissions from the head of the oral care implement. The rinsed surfaces may be exposed to light during or immediately after contact with the rinse, or a time delay of from about 0 seconds to about 2 minutes can occur between rinsing and exposure of the rinsed surfaces to light. Printed instructions can be provided with the packaging that instruct a user to follow any combination of the steps described herein.

In another embodiment, an oral care composition is utilized in conjunction with a tongue device. The composition is preferably applied prior to, concurrently with, or subsequent to, contacting the tongue. Generally, contacting or moving (perhaps without direct contact with the tongue) the tongue device across the surface of the tongue is performed in a period of time from about 0 seconds to about 5 minutes, from about 0 seconds to about 2 minutes, from about 0 seconds to about 1 minute and from about 0 seconds to about 30 seconds. It is also to be noted that the present invention regimens include methods in which an oral care device is used by orienting the device such that light is directed to the regions or surfaces of interest, such as the tongue, without any scraping or contact occurring. In this instance, the light output from the light emitting element is merely directed to the tongue surface for a predetermined period of time sufficient to active the RSG agent across a portion or substantially all of the upper surface of the tongue. Such a strategy could be used as a portion of an overall or larger regimen.

The aforementioned methods can be repeated from about 1, 2, 3, 4 to about 5, 4, 3, 2, 1 times a day for from about 1 day to about 8 weeks. Additionally, the aforementioned methods can be used indefinitely, for example as part of an every day oral care regimen.

Moreover, although the present invention regimen has been primarily described in conjunction with an electric toothbrush having a powered moveable bristle assembly, the invention includes oral care appliances other toothbrushes or other than the toothbrushes described herein. For example, the present invention includes a manual toothbrush used in conjunction with one or more oral care compositions as also described herein.

As previously described, the appliances of the present invention can be used in a variety of ways. As will be appreciated, appliances of the present invention can be used in a traditional brushing regimen, wherein a dentifrice comprising an RSG agent is used and the teeth are brushed in a conventional fashion near the gum line while a light emitting element is energized. A signal might be provided to the user after a predetermined length of time. For instance, a signal might be provided to the user that the light emitting element is on or that it is time to move the brush along the gingival of the upper and lower dentitions. Another signal might be provided to the user indicating that the light emitting element has been turned off. If the rear surface of the brush head incorporates a light emitting element and/or tongue structures, another signal might be provided to indicate that it is time to move the rear surface of the brush head across or near the surface of the tongue. Alterantively, instructions can be provided to the user of the toothbrush with the toothbrush packaging instructing the user to incorporate a step of moving the brush head across the tongue as part of the brushing regimen. In another embodiment of the regimen, a separate composition comprising an RSG agent can be applied to the soft tissue before or after a traditional brushing regimen incorporating a dentifrice. For example, an oral care composition comprising an RSG agent might be applied to the soft tissue, a toothbrush incorporating a light emitting element might then be employed to direct the light output at the soft tissue for a predetermined amount of time sufficient to activate the RSG agent, and then a dentifrice is applied to a toothbrush head and a traditional tooth brushing regimen is employed, perhaps with a different toothbrush head such as a toothbrush head that does not include a light emitting element. The order of these steps may be reversed. Further, a specific step directed to moving the brush head having the light emitting element across the tongue to direct the light onto the tongue for a sufficient period of time to activate the RSG agent may be included at any point in the process. The amount of time required to activate the RSG agent might be less than about 2 minutes, or less than about 90 seconds, or less than about 60 seconds, or less than about 30 seconds and greater than about 5 seconds, or greater than about 10 seconds, or greater than about 15 seconds, or greater than about 20 seconds per dental arch or across the tongue. A timer might be employed to determine when to provide a signal to the user that the predetermined time period has elapsed.

The following patent applications and patents provide further details as to various aspects of the toothbrushes described herein. U.S. application Ser. No. 60/501,266 filed on Sep. 9, 2003; U.S. application Ser. No. 10/832,168 filed on Apr. 26, 2004; U.S. application Ser. No. 10/847,429 filed on May 17, 2004; U.S. application Ser. No. 10/842,302 filed on May 10, 2004; U.S. application Ser. No. 10/887,644 filed on Jul. 9, 2004; U.S. application Ser. No. 10/887,667 filed on Jul. 9, 2004; U.S. application Ser. No. 10/888,206 filed on Jul. 9, 2004; U.S. published application US 2004/0191729A1 filed on Feb. 10, 2004; U.S. published application US 2004/0193235A1 filed on Feb. 10, 2004; U.S. published application US 2004/0193236A1 filed on Feb. 10, 2004; U.S. published application 2004/0199227A1 filed on Feb. 10, 2004; U.S. published application US 2004/0204745A1 filed on Feb. 10, 2004; U.S. published application US 2004/0210276A1 filed on Feb. 10, 2004; and U.S. patent 6,648,904.

Further aspects, details, and variant designs relating to oral care appliances of the present invention are set forth in U.S. Pat. Nos. 3,624,219; 4,066,745; 4,834,969; 5,057,308; 5,057,309; 5,057,310; 5,082,444; 5,095,615; 5,096,699; 6,214,320; and 6,509,007. Published U.S. applications that may also contain similar information include 2001/0002994; 2003/0082113; 2003/0190292; and 2004/0014001 in addition to European publication No. EP 1104669 and international application Serial No. 2005/008050, filed on Mar. 9, 2005. All documents cited herein are, in relevant part, incorporated herein by reference; the citation of any document is not to be construed as an admission that it is prior art with respect to the present invention.

It is significant to note that any of the features, aspects, or details of any method and/or product described herein can be combined, either entirely or partially, with any other feature, aspect, or detail of one or more other methods or products described herein.

All documents cited in the Detailed Description of the Invention are, in relevant part, incorporated herein by reference; the citation of any document is not to be construed as an admission that it is prior art with respect to the present invention. While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of affecting one or more microorganisms in an oral cavity, comprising:
introducing a composition into the oral cavity, wherein said composition comprises a reactive species generating agent; and
directing an output from a light emitting element at a portion of the soft tissue of the oral cavity for a period of time sufficient to generate one or more reactive species from said reactive species generating agent.

2. The method of claim 1, wherein said composition is a rinse.

3. The method of claim 1, wherein said soft tissue comprises the tongue.

4. The method of claim 1, wherein said soft tissue comprises the gingiva.

5. The method of claim 1, further comprising an electric toothbrush having a head and handle, wherein said head comprises said light emitting element.

6. The method of claim 1, wherein said reactive species comprises a reactive oxygen species.

7. The method of claim 5, wherein said light emitting element forms an acute angle with a top surface of said head.

8. The method of claim 5, wherein said light emitting element is disposed in a back surface of said head.

9. The method of claim 8, wherein said back surface of said head further comprises on or more protrusions.

10. The method of claim 9, wherein said one or more protrusions comprise an elastomeric wall.

11. The method of claim 5, further comprising applying a second composition to the oral cavity and brushing one or more teeth.

12. The method of claim 11, wherein said second composition is a dentifrice.

13. The method of claim 11, wherein application of said second composition occurs before application of said first composition.

14. The method of claim 1, wherein said output of said light emitting element is light having an intensity of less than about 100 mW/cm².

15. A method of reducing oral malodor comprising contacting a surface of a tongue with a device comprising one or more protrusions and a light-emitting element.

16. The method of claim 15, wherein said protrusion is an elastomeric wall.

17. A method of reducing oral malodor comprising:
depositing an effective amount of a composition onto a tongue, wherein said composition comprises a reactive species generating agent; and
exposing the tongue to energy from an energy emitting element which activates said reactive species generating agent to produce one or more reactive species.

18. The method of claim 17, wherein said energy is selected from the group consisting of light, heat, electrical energy, vibrational energy, and combinations thereof.

19. The method of claim 17, further comprising brushing the teeth with a second composition and exposing said second composition to light having a wavelength that activates said second composition.

20. The method of claim 17, wherein said reactive species generating agent is selected from the group consisting of thioxanthone, riboflavin, chlorophyll, toludine blue, methylene blue dihaematoporphyrin ester, phthalocyanine metals, metal complexing agents, peroxides, metal chlorites, perporates, percarbonates, peroxyacids, persulfates, and combinations thereof.

21. A method of affecting one or more microorganisms in an oral cavity, comprising:
introducing a composition into the oral cavity, wherein said composition comprises a porphyrin precursor that is used to produce aminolevulinic acid within a microorganism in the oral cavity; and
directing an output from a light emitting element into the oral cavity.

22. A method for affecting a microorganism in an oral cavity, comprising:
introducing a composition into the oral cavity, wherein said composition comprises a porphyrin precursor within a microorganism in the oral cavity; and
directing an output from a light emitting element into the oral cavity.
